(19) United States
(12) Patent Application Publication
(10) Pub. No.: US 2009/0317340 A1
(43) Pub. Date: Dec. 24, 2009

(54) CONCENTRATED BEVERAGE COMPOSITION FOR CLEANING ORAL CAVITY, METHOD OF MANUFACTURING THE CONCENTRATED BEVERAGE COMPOSITION AND NATURAL TEA COMPRISING THE SAME

(76) Inventor: Young Joon Pak, Pyeongtaek-si (KR)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/142,659

(22) Filed: Jun. 19, 2008

(51) Int. Cl. A61K 8/97; A61Q 11/00

(52) U.S. Cl. 424/58

(57) ABSTRACT

Disclosed are a concentrated beverage composition for cleaning an oral cavity, a method of manufacturing the concentrated beverage composition, and a natural tea comprising the same. The concentrated beverage composition may comprise 100 parts by weight of water, 0.1 to 3 parts by weight of a flavoring agent, 1 to 10 parts by weight of mugwort extract, 1 to 15 parts by weight of grapefruit extract, and 1 to 10 parts by weight of radish extract.

### BACKGROUND OF THE INVENTION

1. Field of the Invention

The present invention relates to a beverage composition, a method of manufacturing the beverage composition, and a natural tea comprising the same, and more particularly, to a concentrated beverage composition for cleaning an oral cavity, a method of manufacturing the concentrated beverage composition, and a natural tea comprising the same.

2. Description of Related Art

In general, beverages may contain fruit juices, various additives, and the like, and can relieve people's thirst and instantaneously satisfy a variety of tastes of people using the above ingredients. Recently, as people's interest for health and beauty is increased, functional beverages having added functions such as preventing a hangover, improving blood circulation, increasing beauty and the like as well as an inherent capability of thirst relief are being focused on.

Meanwhile, in order to maintain an oral cavity to be clean, people may typically brush their teeth after meals. However, the brushing may be a troublesome task in view of requiring a toothbrush, toothpaste, and the like.

In this regard, gargling solutions for cleaning the oral cavity by gargling without separate tools have been developed. However, the gargling solutions are required to be spat out after gargling, and causes side effects such as vomiting, diarrhea, and the like when the gargling solutions are swallowed.

### SUMMARY OF THE INVENTION

An aspect of the present invention provides a concentrated beverage composition for cleaning an oral cavity, a method of manufacturing the concentrated beverage composition, and a natural tea comprising the same, which may maintain one's oral cavity to be clean.

According to an aspect of the present invention, there is provided a concentrated beverage composition for cleaning an oral cavity, the composition comprising: 100 parts by weight of water, 0.1 to 3 parts by weight of a flavoring agent, 1 to 10 parts by weight of mugwort extract, 1 to 15 parts by weight of grapefruit extract; and 1 to 10 parts by weight of radish extract.

According to an aspect of the present invention, there is provided a method for manufacturing a concentrated beverage composition for cleaning an oral cavity, the method comprising: preparing a flavoring agent, preparing a mixture of raw sources and water, the raw sources comprising mugwort, grapefruit, and radish, preparing an extract solution comprising mugwort extract, grapefruit extract, and radish extract using the prepared mixture, preparing a mixed solution by mixing the prepared flavoring agent and the extract solution; and preparing a concentrated solution by concentrating the prepared mixture.

According to an aspect of the present invention, there is provided a natural tea for cleaning an oral cavity, the natural tee comprising: a concentrated beverage composition for cleaning an oral cavity comprising 100 parts by weight of water, 0.1 to 3 parts by weight of a flavoring agent, 1 to 10 parts by weight of mugwort extract, 1 to 15 parts by weight of grapefruit extract, and 1 to 10 parts by weight of radish extract; and 300 to 900 parts by weight of ion water comprising germanium, based on 100 parts by weight of the concentrated beverage composition.

### DETAILED DESCRIPTION OF EXEMPLARY EMBODIMENTS

Hereinafter, a concentrated beverage composition for cleaning an oral cavity, a method of manufacturing the concentrated beverage composition, and a natural tea comprising the same according to the present invention will be described in detail.

The concentrated beverage composition for cleaning the oral cavity may be separately drunk, however the present invention is not limited thereto. As an example, the concentrated beverage composition for cleaning the oral cavity may be mixed with ion water comprising germanium to thereby manufacture a natural tea for drink. In addition, the concentrated beverage composition for cleaning the oral cavity may be added to a variety of other beverages. Herein, various additives may be added to the natural tea and the variety of other beverages depending on people's preference.

The concentrated beverage composition for cleaning the oral cavity according to the present invention may comprise 100 parts by weight of water, 0.1 to 3 parts by weight of a flavoring agent, 1 to 10 parts by weight of mugwort extract, 1 to 15 parts by weight of grapefruit extract, and 1 to 10 parts by weight of radish extract. Also, the concentrated beverage composition for cleaning the oral cavity according to the present invention may further comprise 0.5 to 3 parts by weight of pine needle extract. In addition, the concentrated beverage composition for cleaning the oral cavity may further comprise other additives, etc. for the purpose of improving taste and flavor of the concentrated beverage composition for cleaning the oral cavity. The other additives may comprise fruit juices such as peach juice, lemon juice, and the like, and comprise 1 to 10 parts by weight of the fruit juice based on 100 parts by weight of water.

Hereinafter, each ingredient of the concentrated beverage composition for cleaning the oral cavity according to the present invention will be described in detail. The natural ingredients may be subjected to a hot water extraction method under high pressure to obtain extracts of the natural ingredients, thereby acquiring function and efficacy of the natural ingredients. Accordingly, the natural ingredients will be hereinafter described in detail.

The flavoring agent may comprise a natural or artificial flavoring agent, which is well known to skilled persons in the art. As an example, the flavoring agent may at least one essential oil extracted from herbs comprising clove, peppermint, spearmint, jasmine, rosemary, and Eucalyptus. Preferably, the flavoring agent may comprise essential oil extracted from clove, peppermint, and jasmine, respectively. Also, the flavoring agent may further comprise at least one essential oil extracted from fruits comprising apple, pear, peach, strawberry, cherry, plum, pineapple, and apricot, and/or seeds of the fruits. Preferably, the flavoring agent may further comprise essential oil extracted from a seed of apricot. In addition, various flavoring agent may be used.

Herein, the essential oil may denote a high concentrated stock solution extracted from flowers, leaves, stems, roots, seeds and the like of aromatic plants. The essential oil may be extracted by any one of a hot water extraction scheme method, a steam distillation method, an absorption method, a solvent extraction method, and an exudation method, however, the present invention is not limited thereto.

Mugwort may be a perennial plant having a relatively strong propagation which is distributed widespread in Asia such as South Korea, Japan, and China, Europe, and the like, and belong to Carduaceae. Mugwort may have effects on lower abdominal pain, hookworm, removal of offensive odor, gastroenteric disorder, constipation, neuralgia, cryopathy, and gynecopathy as well as hemostasis and digestion promotion. The mugwort may have various biological effects such as antimicrobial effect, insecticidal effect, antineoplastic effect, and the like due to its aromatic ingredients or ethereal oil ingredients. Also, extract obtained by performing the hot water extraction with respect to the mugwort may have a significantly strong monovalence with respect to Streptococcus mutans, Staphylococcus aureus, and Candida albicans, which are main infectious strains within the oral cavity.

Grapefruit is a type of tropical fruit, and may have effects on maintaining liver, stomach, internal organs, and the like to be cleaned, and promoting excretion. In particular, an extract solution of a grapefruit seed may be subacid, and have antibacterial effect in acidity.

Radish may comprise a large amount of vitamin C, benefit liver, heart, spleen, lung, and kidney, and help refection. Also, radish may remove bacteria within the stomach, promote digestion, alleviate coughs and fevers due to colds, and the like.

Pine needles may be referred to as leaves of a pine tree, and whose main ingredients are Terpentine oil, Cineole, Salinigrin, Coniferin, P-Cymen, Densipimaric acid, Retene, protein, phosphorus (P), iron (Fe), minerals, fat soluble vitamin A and vitamin C, and the like. Pine needles may have effects on hypertension, arrhythmia, and the like, and also prevent cohesion of the blood.

The concentrated beverage composition for cleaning the oral cavity according to the present invention comprising the above-described flavoring agent and natural ingredients may prevent halitosis through drinking the same. Specifically, the concentrated beverage composition for cleaning the oral cavity according to the present invention may maintain the oral cavity to be clean regardless of time and location.

Hereinafter, a method for manufacturing a concentrated beverage composition for cleaning the oral cavity according to the present invention will be described in detail.

The method for manufacturing the concentrated beverage composition for cleaning the oral cavity may comprise preparing a flavoring agent, preparing a mixture of raw sources and water, the raw sources comprising mugwort, grapefruit, and radish, preparing an extract solution comprising mugwort extract, grapefruit extract, and radish extract using the prepared mixture, preparing a mixed solution by mixing the prepared flavoring agent and the extract solution, and preparing a concentrated solution by concentrating the prepared mixture.

Herein, the flavoring agent may comprise at least one essential oil extracted from herbs comprising clove, peppermint, spearmint, jasmine, rosemary, and Eucalyptus. Also, the flavoring agent may further comprise at least one essential oil extracted from fruits comprising apple, pear, peach, strawberry, cherry, plum, pineapple, and apricot, and/or seeds of the fruits.

First, the preparing of the flavoring agent will be herein described in detail. For example, clove, peppermint, jasmine, and apricot stone are cleaned by washing, and the washed ingredients of the flavoring agent are put into a flexible pouch. Next, water of a 1 to 50 times the amount relative to the flavoring agent at about 20° C. to about 50° C. is added in the pouch, and then the pouch is sealed. Next, the ingredients of the flavoring agent within the pouch are subjected to a hot water extraction at about 80° C. to about 100° C. for about 30 to about 200 minutes to obtain the flavoring agent. Then, ingredients, not being subjected to the hot water extraction, from among the ingredients of the flavoring agent are filtered, thereby preparing the flavoring agent.

Next, mugwort, grapefruit, and radish are cleaned by washing, and the washed sources are put into another flexible pouch. Next, water of a 1 to 20 times the amount relative to the washed sources at about 0° C. to 50° C. is added in the pouch, and then the pouch is sealed, thereby preparing the mixture.

Next, the preparing of the extraction solution may comprise pressing and filtering the prepared mixture. As an example, the pouch is pressed with about 100 to about 600 MPa pressure at about 80° C. to about 100° C. for about 30 to about 300 minutes. In these processes, the raw sources are subjected to the hot water extraction to obtain each of extracts, that is, mugwort extract, grapefruit extract, and radish extract. Next, the raw sources, not being subjected to the hot water extraction, from among the mixture may be filtered. Herein, a typical hot water extraction scheme instead of the above-described hot water extraction scheme using high pressure may be used, and also other various schemes may be used.

Next, the prepared flavoring agent and the prepared extraction solution are mixed together, thereby preparing the mixture solution.

Next, the prepared mixture is concentrated, thereby preparing the concentrated solution. The preparing of the concentrated solution prepares the concentrated solution such that the weight of the concentrated solution is about 5 to about 20% of the weight of the mixture. As an example, the prepared extract solution is heated to about 80° C. to about 100° C. for about 30 to about 200 minutes, thereby controlling the weight of the concentrated solution. The concentrated solution prepared after the above-described process is performed may comprise the flavoring agent, extracts of the natural sources, and water, and each content of the extracts is the same as the above.

The concentrated beverage composition for cleaning the oral cavity according to the present invention may prevent halitosis, thereby maintaining the oral cavity to be clean regardless of time and location

Meanwhile, a natural tea for cleaning an oral cavity according to the present invention will be herein described in detail.

The natural tea for cleaning the oral cavity according to the present exemplary embodiment of the invention may comprise 100 parts by weight of water, 0.1 to 3 parts by weight of a flavoring agent, 1 to 10 parts by weight of mugwort extract, 1 to 15 parts by weight of grapefruit extract, and 1 to 10 parts by weight of radish extract, and 300 to 900 parts by weight of ion water comprising germanium, based on 100 parts by weight of the concentrated beverage composition. Also, the natural tea for cleaning the oral cavity according to the present invention may further comprise 1 to 10 parts by weight of honey, based on 100 parts by weight of the concentrated beverage composition, and 15 to 50 parts by weight of zinc salt. Herein, the concentrated beverage composition is the same as the above, and thus repeated descriptions will be herein omitted.

The natural tea may comprise ingredients other than the above-described ingredients depending on people's preference. As an example, ingredients having a sour taste such as a lemon juice and a sweet taste such as sugar may be further added in the natural tea.

The ion water comprising germanium may have efficacy of the germanium. Specifically, the germanium may supplement oxygen to the human body to have antioxidant action, thereby preventing acidification of blood in the human body, and preventing aging of cells. Also, the germanium may combine with wastes within the blood vessel, such as cholesterol, lipid, thrombus, and the like to thereby cleanse the blood, improve circulation of the blood, and control blood pressure. As a result, the germanium may combine with various heavy metals such as mercury (Hg), cadmium (Cd), lead (Pb), and the like, so that the combined heavy metals can be expelled to the outside of the human body. Also, the germanium may have superior antiviral action and analgesic action, and promote absorption of minerals such as calcium (Ca), zinc (Zn), and the like, thereby improving osteoporosis. Accordingly, by drinking the natural tea for cleaning an oral cavity, overall improvement of health status due to the germanium as well as cleaning an oral cavity may be expected.

The ion water comprising the germanium may further comprise other ingredients other than the germanium. As an example, the ion water comprising the germanium may comprise at least one from a group consisting of potassium (K), silica, sodium (Na), calcium (Ca), chlorine (Cl), carbonic acid, fluorine (F) in very small amounts. In addition, the ion water comprising the germanium may further comprise other ingredients comprised of general ion water.

Honey may comprise a heavy solution of monosaccharide having a floral aroma. A solid content of honey is about 80 wt %, and a moisture content of the same is about 17 wt %. Fructose and glucose are mainly comprised of the solid content of honey. Honey may have a high viscosity and sticky characteristics due to content of the monosaccharide, and also have a sweet taste.

The zinc salt may have antimicrobial effect. The zinc salt may use at least one of a group consisting of zinc chloride, zinc sulfate, zinc nitrate, zinc citrate, zinc gluconate, zinc acetate, zinc lactic acid, and zinc salicylic acid. Preferably, the zinc chloride may be used.

Hereinafter, the present invention will be described in detail by examples. It is to be understood, however, that these examples are for illustrative purpose only, and are not construed to limit the scope of the present invention.

### EXAMPLES

### Example 1

### Manufacturing of a Concentrated Beverage Composition for Cleaning the Oral Cavity

10 g of clove, 10 g of peppermint, 30 g of jasmine, and 5 g of apricot stone were cleaned by washing, and then put into a pouch. Next, 1 kg of water at 25° C. was added in the pouch, and the pouch was sealed. Next, the raw sources within the pouch were subjected to a hot water extraction to obtain the essential oil at about 95° C. for about 100 minutes. The raw sources, not being subjected to the hot water extraction, were filtered, thereby preparing 5 g of the flavoring agent.

Next, 120 g of mugwort, 150 g of grapefruit, and 110 g of radish were cleaned by washing, and then put into another pouch. Next, 1 kg of water at 25° C. was added in the pouch, and the pouch was sealed, thereby preparing the mixture solution.

Next, the pouch was pressed with about 500 MPa pressure at about 90° C. for about 100 minutes, and then the mixture solution was subjected to the hot water extraction to obtain the mugwort extract, the grapefruit extract, and the radish extract. Next, the mixture solution, not being subjected to the hot water extraction, from among the mixture solution was filtered, thereby preparing 690 g of the extraction solution.

Next, 5 g of the flavoring agent and 690 g of the extraction solution were mixed together, thereby preparing 695 g of the mixture solution.

Next, the extraction solution was heated at about 90° C. for about 120 minutes, thereby preparing 120 g of the concentrated solution, and thus manufacturing the concentrated beverage composition for cleaning the oral cavity.

### Example 2

### Manufacturing of the Natural Tea for Cleaning the Oral Cavity

120 g of the concentrated beverage composition was added in 1 kg of ion water comprising germanium, and 40 g of honey and 20 g of zinc salt were added in the ion water, thereby manufacturing the natural tea for cleaning the oral cavity.

### Estimation of Oral Cavity Status

After 50 adults drank the manufactured natural tea, a survey with respect to cleaning of the oral cavity was carried out, and the results of the sensory test are shown in the following Table 1.

TABLE 1

| | Very good | Good | Medial | No change |
|---|---|---|---|---|
| feeling of | 24 | 15 | 9 | 2 |
| refreshment | | | | |
| Removal of | 10 | 32 | 7 | 1 |
| halitosis | | | | |

As can be seen in the above Table 1, the natural tea for cleaning the oral cavity may provide a feeling of refreshment and have effects on removal of halitosis.

As described above, according to the present invention, the concentrated beverage composition for cleaning the oral cavity comprising the flavoring agent, the mugwort extract, the grapefruit extract, and the radish extract may be drunk alone, or a natural tea manufactured using the concentrated beverage composition may be drunk, thereby preventing halitosis, and also maintaining the oral cavity to be cleaned regardless of time and location.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

1. A concentrated beverage composition, concentrated from a prepared mixture for cleaning an oral cavity, the concentrated composition comprising:
100 parts by weight of water;
0.1 to 3 parts by weight of a flavoring agent;
1 to 10 parts by weight of mugwort extract;
1 to 15 parts by weight of grapefruit extract; and
1 to 10 parts by weight of radish extract
wherein the concentrated beverage composition is a concentrated solution with a weight equal to about 5% to about 20% of the weight of the prepared mixture; and wherein the concentrated beverage composition is prepared by a removal of water from the prepared mixture.

2. The composition of claim 1, wherein the flavoring agent comprises at least one essential oil extracted from herbs comprising clove, peppermint, spearmint, jasmine, rosemary, and Eucalyptus.

3-9. (canceled)

10. A natural tea for cleaning an oral cavity, the natural tee comprising:
a concentrated beverage compositions concentrated from a prepared mixture, for cleaning an oral cavity comprising 100 parts by weight of water, 0.1 to 3 parts by weight of a flavoring agent, 1 to 10 parts by weight of mugwort extract, 1 to 15 parts by weight of grapefruit extract, and 1 to 10 parts by weight of radish extract; and
300 to 900 parts by weight of ion water comprising germanium, based on 100 parts by weight of the concentrated beverage composition
wherein the concentrated beverage composition is a concentrated solution with a weight equal to about 5% to about 20% of the weight of the prepared mixture; and wherein the concentrated beverage composition is prepared by removal of water from the prepared mixture.

11. (canceled)

12. The natural tea of claim 10, wherein the flavoring agent of the concentrated beverage composition comprises at least one essential oil extracted from herbs comprising clove, peppermint, spearmint, jasmine, rosemary, and Eucalyptus.

13-19. (canceled)
